(19) United States
(12) Patent Application Publication
(10) Pub. No.: US 2015/0011981 A1
(43) Pub. Date: Jan. 8, 2015

(54) Catheter Manufacture

(71) Applicant: Adapta Medical, Inc., Colorado Springs, CO (US)

(72) Inventor: Jamie Glen House, Colorado Springs, CO (US)

(73) Assignee: Adapta Medical, Inc., Colorado Springs, CO (US)

(21) Appl. No.: 14/326,066

(22) Filed: Jul. 8, 2014

Related U.S. Application Data
(60) Provisional application No. 61/843,851, filed on Jul. 8, 2013.

(51) Int. Cl. A61M 25/00; A61J 1/10

(52) U.S. Cl. CPC A61M 25/0009; A61M 25/0017; A61J 1/10; A61M 2025/0004
USPC 604/544; 264/163; 264/249

(57) ABSTRACT

Devices and methods are disclosed relating to a combination sheath and collection bag for a catheter assembly. A catheter sheath and a collection bag are manufactured as one receptacle. Though in early phases of manufacture the catheter sheath and collection bag are in fluid communication, the two are partitioned before the catheter assembly is complete.

This U.S. Patent Application claims priority to U.S. Provisional Patent Application Ser. No. 61/843,851, filed Jul. 8, 2013, the content of which is hereby incorporated by reference herein in its entirety into this disclosure.

### BACKGROUND OF THE SUBJECT DISCLOSURE

1. Field of the Subject Disclosure

The present subject disclosure relates to urinary catheters. More specifically, the present subject disclosure relates to the manufacturing of a catheter for a person with limited manual dexterity.

2. Background of the Subject Disclosure

Short term, or repeated catheterization of an individual's urinary bladder is a common practice today for many persons who are in a hospital setting, a nursing home, doctor's office, rehabilitation facility or at home. For instance, a user is sometimes catheterized to treat such conditions as urinary retention, the inability to evacuate urine, or for the purpose of obtaining a sterile urine specimen from a user in a doctor's office.

The need for intermittent catheterization of an individual sometimes arises due to problems typically associated with long term use of indwelling catheters, such as infections, urethral damage, and bladder damage. Long term use of an indwelling catheter is also a risk factor for bladder cancer. This is often the case for persons having a neurogenic urinary condition, such as in a spinal cord injury, multiple sclerosis, stroke, trauma or other brain injury. Conditions that interfere with the individual's ability to voluntarily void the bladder may also arise post-surgically or as a result of benign prostatic hypertrophy or diabetes. Many of the affected individuals are capable of, and would prefer to perform self-catheterization. For many, the level of risk and discomfort of repeated catheterizations carried out over the course of a day (at 3-6 hour intervals, for example) are offset by the accompanying convenience, privacy or self-reliance that is achieved. Some of the major difficulties that arise in self-catheterization are the lack of satisfactory catheterization kits, the problem of maintaining the required level of sanitation during the procedure, and the difficulty of sometimes performing the procedure under conditions of restricted space and privacy.

In assisted, or non self-catheterizations, it is common practice in hospitals to employ a catheterization tray, which typically includes a sterile drape, gloves, a conventional catheter, antiseptic solution, swabs, lubricant, forceps, underpad and a urine collection container. Assisted catheterization is usually performed with the user in a supine position. Maintaining a sterile field during the procedure can still be a problem, however, and the “cath tray” procedure is impractical for use with some individuals and situations today.

Many individuals with spinal cord injuries or other neurological diseases routinely perform intermittent catheterization several times a day using conventional catheters or kits and “clean technique.” Clean technique means that the urethral area is initially swabbed with antiseptic, and efforts are made to avoid contamination of the catheter during the procedure. The user's hands are not sterile and a sterile field is not maintained. Clean technique is used instead of sterile technique, generally, for two reasons. First, it is very difficult, if not impossible, for individuals who are performing self-catheterization to adhere strictly to sterile technique. Secondly, these individuals are required to catheterize themselves between 3 and 6 times a day, and the cost of a new sterile catheter and the accessories required to perform sterile catheterization become excessively expensive for many users. Sometimes an individual will reuse a “cleaned” catheter. As a result, the use of non-sterile technique will many times result in contamination and subsequent infection of the urinary tract, causing significant morbidity and cost to the user and society.

Even if cost considerations were not a major consideration for the user, with most conventional self-contained sterile units where the collection bag doubles as the catheter insertion cover, the catheter is extremely difficult for the user to grasp and insert. This is particularly a problem for self-catheterization users who may also have neurological problems that limit manual dexterity. Also, with some of the available catheter kits and methods, the catheter is either not sufficiently lubricated during insertion (and thus requires the additional application of possibly non-sterile lubricant), or the catheter is too slick with lubricant and cannot effectively be grasped through an insufficiently flexible bag. As a practical matter, most individuals who would prefer to self-catheterize cannot conveniently do so, and maintain the required level of sanitation using many of the existing catheterization apparatus.

Many catheterization tasks require a degree of dexterity to accomplish. People with normal dexterity, like paraplegics, may not have use of their lower extremities, but their hands are normal. On the other hand, quadriplegics can have use of their upper extremities, having absolutely normal movement, like a paraplegic, except for normal hand dexterity. Thus, many tasks requiring a degree of hand dexterity are very difficult for quadriplegics to accomplish.

Spinal cord injuries at the C5, C6, or C7 level often affect the use of a person's hands and make these tasks difficult. However, people who have had strokes, brain injuries, or multiple sclerosis may also require catheterization but have limited dexterity. In this, and other ways, the current catheterization market does not currently support the needs of these people.

Manufacture of the catheter sheath and collection bag separately requires a process and machine for making each, then another process and machine for combining the two, which drives up costs.

### SUMMARY OF THE SUBJECT DISCLOSURE

The present subject disclosure solves the problem described above by manufacturing a combination sheath and collection bag for a catheter assembly. In exemplary embodiments of the present subject disclosure, a catheter sheath and collection bag, both of which are receptacles, are manufactured as one receptacle. Though in early phases of manufacture the catheter sheath and collection bag are in fluid communication, the two are partitioned before the catheter assembly is complete. In one exemplary embodiment, the partition is made by pressing the area of the sheath just proximal to the distal end of a catheter after the catheter has been inserted into the sheath. The partition anchors the distal end of the catheter to prevent the catheter from falling out of an introducer housing the proximal end of the catheter. In another exemplary embodiment, shrink wrap or other heat sealant is wrapped around the sheath just proximal to the distal end of the inserted catheter to provide for both the partitioning of the catheter sheath and the collection bag as well as anchoring the distal end of the catheter.

In one exemplary embodiment, the present subject disclosure is a urinary catheterization assembly. The assembly includes a catheter having a proximal end and a distal end, a sheath substantially covering the catheter, a collection bag attached to the distal end of the catheter, such that a fluid traveling through the catheter is securely collected in the collection bag. The sheath and the collection bag are manufactured as one receptacle, then partitioned just proximal to the distal end of the catheter.

In another exemplary embodiment, the present subject disclosure is a method for manufacturing a urinary catheterization assembly. The method includes cutting a single receptacle comprising a sheath portion and a collection bag, and stamping a partition at a region where the sheath portion meets the collection bag. A catheter may be substantially covered by the sheath portion. The partition may be positioned just proximal to a distal end of the catheter.

In yet another exemplary embodiment, the present subject disclosure is A urinary catheterization assembly, including a sheath substantially covering a catheter, and a collection bag attached to a distal end of the catheter, such that a fluid traveling through the catheter is securely collected in the collection bag. The sheath and the collection bag are manufactured as one receptacle.

### BRIEF DESCRIPTION OF THE DRAWINGS

FIG. 1 shows a catheter assembly having a sheath and collection bag manufactured as one receptacle, according to an exemplary embodiment of the subject disclosure.

FIG. 2 shows another catheter assembly, according to an exemplary embodiment of the subject disclosure.

FIG. 3 shows a pair of each of a sheath and collection bag manufactured from a single piece, according to an exemplary embodiment of the subject disclosure.

### DETAILED DESCRIPTION OF THE SUBJECT DISCLOSURE

The present application refers to subject matter described in commonly-owned U.S. Pat. No. 6,090,075, issued on Jul. 18, 2000, the content of which is hereby incorporated by reference herein in its entirety into this disclosure.

The subject disclosure relates to a urinary catheterization assembly including a catheter having a proximal and a distal end, a sheath covering the catheter, and a collection bag attached to the distal end of the catheter such that the fluid traveling through the catheter is securely collected in the collection bag. More specifically, the present subject disclosure describes a sheath and a collection bag being manufactured as one receptacle. Manufacturing the sheath and the collection bag from a single piece of material reduces the costs and number of operations required. For instance, fewer processes and materials associated with the manufacturing of the urinary catheterization assembly are required, consequently lowering manufacturing expenses. Further, a sheath and collection bag manufactured as one receptacle results in fewer moving parts, and increased durability of the final product. Other elements may be incorporated into the catheter, and are described in U.S. Pat. No. 6,090,075.

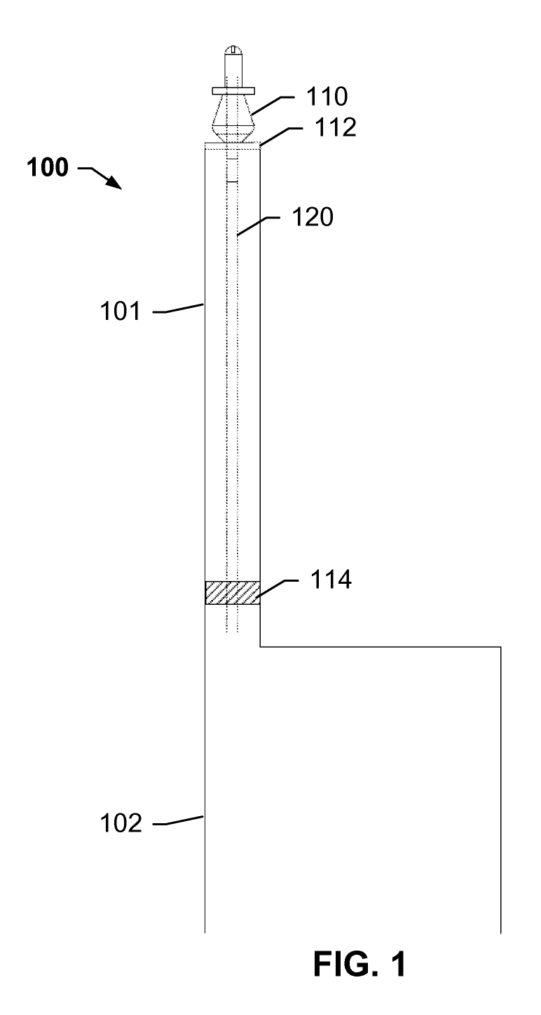

FIG. 1 shows a urinary catheter assembly 100, including an introducer 110 comprising a reservoir and a guide portion, a catheter 120, and a receptacle making up a sheath 101 and a collection bag 102. The sheath portion 101 of the receptacle surrounds all but the proximal and distal end of catheter 120. The proximal end of catheter 120 rests inside the reservoir of guide portion 110 and is held in place by base portion 112 of guide portion 110. The reservoir holds lubricant for lubricating catheter 120 prior to insertion. An introducer is coupled to the proximal end of guide portion 110, and may be sealed prior to use. The guide portion 110 is coupled to the proximal end of the sheath 101 at base portion 112. The sheath 101 and the collection bag 102 of the urinary catheterization assembly are manufactured as one receptacle.

As shown in FIG. 1, the area of the sheath 101 just proximal to the distal end of catheter 120 is partitioned so that collection bag 102 and sheath 101 are not in fluid communication. The partitioning of this area prevents any potential flow of the fluid collected in collection bag 102 back into sheath 101. The partitioning of sheath 101 from collection bag 102 can be achieved in various ways, such as using a hot press to bind the receptacle material. In addition, the distal end of catheter 120 may be bound in place by the hot press. For instance, a flange 114 may be used to seal or partition sheath 101 from collection bag 102, and to anchor catheter 120. Anchoring catheter 120 at its distal end prevents the proximal end of the catheter from falling out of the introducer.

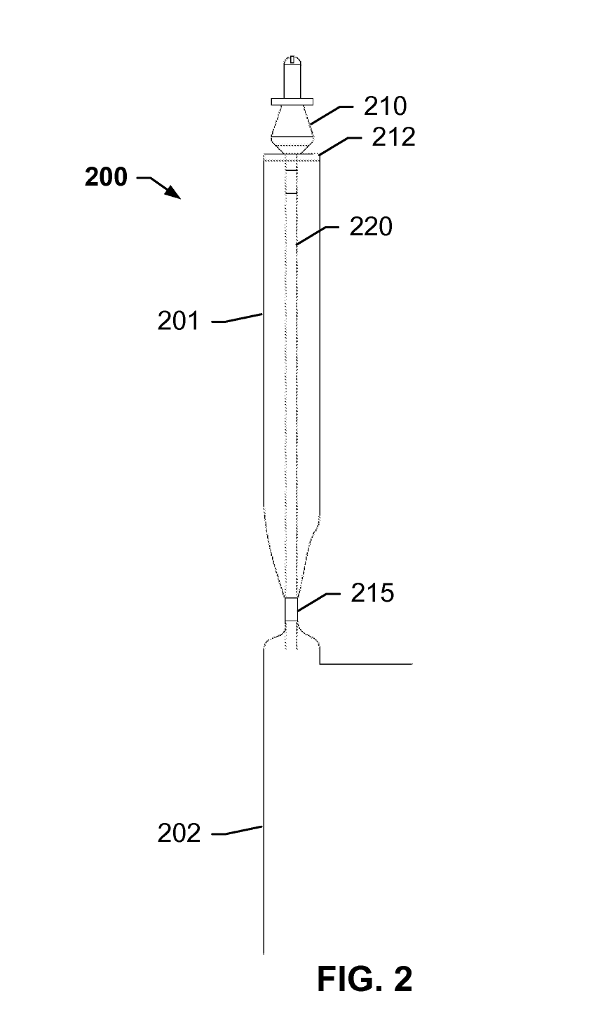

FIG. 2 shows another catheter assembly, according to an exemplary embodiment of the subject disclosure. Similar to FIG. 1, a urinary catheter assembly 200 includes an introducer 210 comprising a reservoir and a guide portion, a catheter 220, and a receptacle making up a sheath 201 and a collection bag 202. The sheath portion 201 of the receptacle surrounds all but the proximal and distal end of catheter 214. The proximal end of catheter 214 rests inside the reservoir of guide portion 210 and is held in place by base portion 212 of guide portion 210. The guide portion 210 is coupled to the proximal end of the sheath 201 at base portion 212.

Similar to the catheter assembly of FIG. 1, sheath 201 and collection bag 202 of the urinary catheterization assembly are manufactured as one receptacle, and the area of sheath 201 just proximal to the distal end of catheter 220 is partitioned so that collection bag 202 and sheath 201 are not in fluid communication. However, in this embodiment, the partition may be formed by a seal 215 that prevents any potential flow of the fluid collected in collection bag 202 back into sheath 201, and binds the distal end of catheter 201 in place. The seal also serves to anchor catheter 220 at its distal end prevents the proximal end of the catheter from falling out of the introducer.

In additional exemplary embodiments, the partitioning may be achieved by placing a shrink wrap around the area of the sheath just proximal to the distal end of the catheter after the catheter has been inserted into the sheath. In addition, the distal end of the catheter is bound in place by the shrink wrap. Anchoring the catheter at its distal end prevents the proximal end of the catheter from falling out of the introducer. In yet another exemplary embodiment, the partitioning can be achieved by using an adhesive inside the area of the sheath just proximal to the distal end of the catheter after the catheter has been inserted into the sheath. In addition, the adhesive also anchors the distal end of the catheter, preventing the proximal end of the catheter from falling out of the introducer.

Moreover, the distal end of the catheter may be anchored so as to keep it within the introducer in the guide and ready to be used. This works for either gel lubricants or for hydrophilic catheters with a small cache packet of saline loose in the sheath. The packet may remain in the guide portion during use and after it is punctured or smashed, releasing the saline. The saline may initially be released from the cache and flow over the catheter, lubricating the catheter prior to insertion. The catheter may then be held upright and the saline in the sheath drains into the urine collection bag. Therefore, the attachment of the distal end of the catheter at the junction of the narrow sheath and large urine collection bag prevents the catheter from falling into the bag and keeps the proximal end in the guide, while maintaining some space open for fluid communication between the sheath and urine collection bag so the saline in the sheath is able to flow into the urine collection bag.

Moreover, the receptacle, which makes up the sheath and the collection bag, may be manufactured in many ways. In one exemplary embodiment, the receptacle is manufactured by hot pressing sheets of material around a perimeter. Once pressed, the excess material is trimmed and either recycled or disposed. Also, in many embodiments the collection bag is wider than the sheath. In order to decrease the amount of recycled and wasted material, the sheath portion of the receptacle is placed substantially to one side of the collection bag portion of the receptacle.

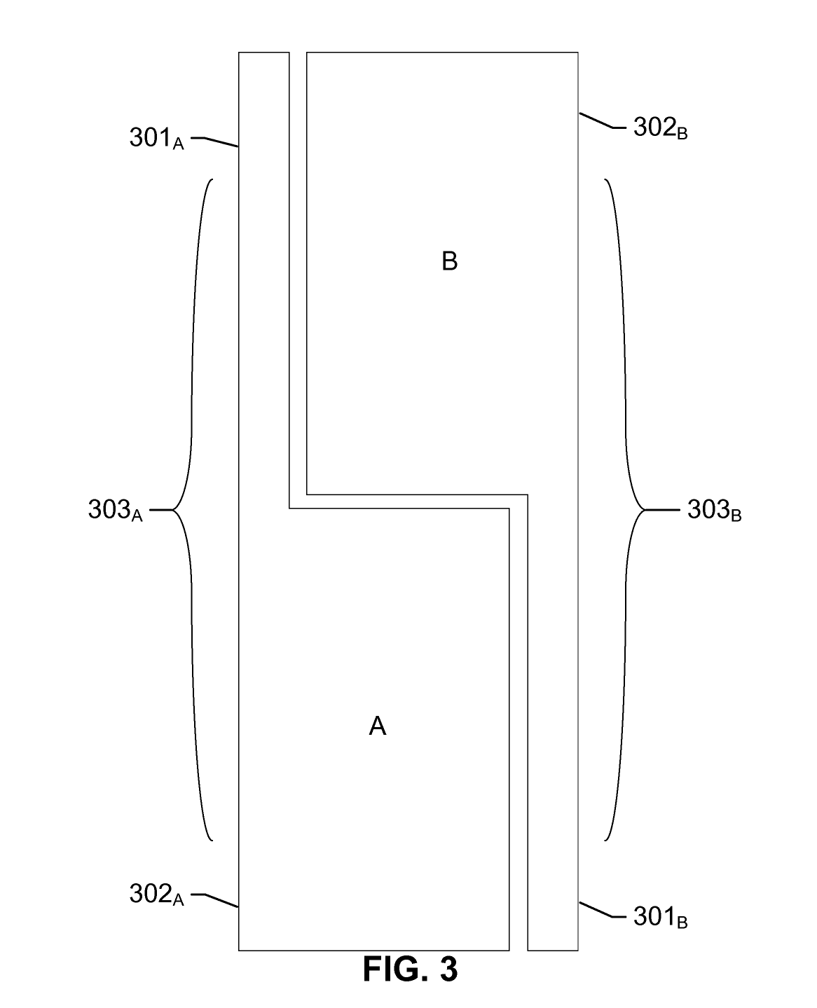

FIG. 3 shows a pair of receptacles manufactured from a single rectangle of material. For instance, receptacles 303_{A} and 303_{B} may be cut from the same piece of material, or pressed using a hot press, with minimal excess material to be recycled or wasted. Each of receptacles 303_{A} and 303_{B} include sheath portions 301 _{A} and 301 _{B} and collection bag portions 302_{A} and 302_{B}, respectively. Enabling the receptacles to be made from one piece ensures minimal wastage of material and minimal process steps, thereby reducing costs and complications.

The sheath and the collection bag may be manufactured from material including but not limited to polyethylene, polypropylene, etc. Furthermore, the collection bag can take on various shapes or forms. The collection bag can have a square shape as depicted in FIGS. 1-3, a circular shape, or any other shape that permits labeling of the collection bag for measurement purposes.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A urinary catheterization assembly, comprising:
a catheter having a proximal end and a distal end;
a sheath substantially covering the catheter; and
a collection bag attached to the distal end of the catheter, such that a fluid traveling through the catheter is securely collected in the collection bag;
wherein the sheath and the collection bag are manufactured as one receptacle, then partitioned just proximal to the distal end of the catheter.

2. The assembly of claim 1, wherein the partition is stamped into the receptacle while the catheter is placed inside the sheath.

3. The assembly of claim 1, wherein the partition includes shrink wrap.

4. The assembly of claim 1, further comprising an introducer at the proximal end of the sheath.

5. The assembly of claim 4, wherein the introducer houses the proximal end of the catheter.

6. The assembly of claim 1, wherein the partition anchors the catheter such that the proximal end of the catheter is prevented from falling out of the introducer.

7. A method for manufacturing a urinary catheterization assembly, comprising:
cutting a single receptacle comprising a sheath portion and a collection bag; and
stamping a partition at a region where the sheath portion meets the collection bag;
wherein a catheter is substantially covered by the sheath portion; and
wherein the partition is just proximal to a distal end of the catheter.

8. The method of claim 7, further comprising stamping the partition into the receptacle while the catheter is placed inside the sheath.

9. The method of claim 7, wherein the cutting further comprises hot pressing sheets of material around a perimeter.

10. The method of claim 9, further comprising trimming excess material around the perimeter.

11. The method of claim 7, wherein the sheath portion of the receptacle is placed substantially to one side of the collection bag portion of the receptacle.

12. A urinary catheterization assembly, comprising:
a sheath substantially covering a catheter; and
a collection bag attached to a distal end of the catheter, such that a fluid traveling through the catheter is securely collected in the collection bag;
wherein the sheath and the collection bag are manufactured as one receptacle.

13. The assembly of claim 12, wherein the sheath and the collection bag are partitioned just proximal to the distal end of the catheter.

14. The assembly of claim 13, wherein the partition is stamped into the receptacle while the catheter is placed inside the sheath.

15. The assembly of claim 12, wherein the partition includes shrink wrap.

16. The assembly of claim 12, further comprising an introducer at the proximal end of the sheath.

17. The assembly of claim 16, wherein the introducer houses the proximal end of the catheter.

18. The assembly of claim 12, wherein the partition anchors the catheter such that the proximal end of the catheter is prevented from falling out of the introducer.
